(19) United States
(12) Patent Application Publication
(10) Pub. No.: US 2013/0196874 A1
(43) Pub. Date: Aug. 1, 2013

(54) SENSOR FOR DETECTION AND IDENTIFICATION OF ANALYTES AND A METHOD THEREOF

(71) Applicants: Ron Naaman, Yarkona (IL); Amos Bardea, Netanya (IL)

(72) Inventors: Ron Naaman, Yarkona (IL); Amos Bardea, Netanya (IL)

(73) Assignee: YEDA RESEARCH AND DEVELOPMENT CO. LTD., Rehovot (IL)

(21) Appl. No.: 13/816,185

(22) PCT Filed: Aug. 4, 2011
(86) PCT No.: PCT/IL11/00635

§ 371 Date: Apr. 15, 2013

Related U.S. Application Data
(60) Provisional application No. 61/371,872, filed on Aug. 9, 2010.

(51) Int. Cl. G01N 33/53

(52) U.S. Cl. CPC G01N 33/5306
USPC 506/9; 506/2

(57) ABSTRACT

The present invention discloses a method and sensor for detection and identification of analytes such as foreign material or DNA and RNA in particular in low concentration. The sensitivity of the method allows the detection of few molecules (down to a single one) independent on the concentration of the sample. There is provided a sensor for analyte(s) detection in a fluid sample. The sensor comprises at least one tube-like member carrying an arrangement of probes patterned thereinside.

### FIELD OF THE INVENTION

This invention relates to a sensor for use in the detection and identification of analytes and a method for detection thereof.

### REFERENCES

1. Marshall, A., J. Hodgson. (1998). DNA chips: an array of possibilities. Nat. Biotechnol. 16:27-31.

2. A. Halperin, A. Buhot, E. B. Zhulina, Sensitivity, Specificity, and the Hybridization Isotherms of DNA Chips, Biophysical Journal, 86, 718-730 (2004).

3. T. G. Drummond, M. G. Hill, J. K. Barton, Electrochemical DNA sensors, Nature Biotechnology, 21, 1192 (2003).

4. Branton, D. et al. (2008) The potential and challenges of nanopore sequencing. Nature Biotechnology 26, 1146-1153.

5. Thewes, R. et al. (2002) Sensor arrays for fully-electronic DNA detection on CMOS. IEEE International Solid-State Circuits Conference. Digest of Technical Papers (Cat. No. 02CH37315), vol. 1 350-473, vol. 2 500-600.

6. M. Kubista, J. M. Andradeb, M. Bengtssona, A. Forootand, J. Jonáke, K. Linda, R. Sindelkae, R. Sjöbacka, B. Sjögreend, L. Strömboma, A. Ståhlberga, N. Zorica, The real-time polymerase chain reaction, Molecular Aspects of Medicine 27 (2006) 95-125.

7. E. T. Lagally, I. Medintz, R. A. Mathies, Single-Molecule DNA Amplification and Analysis in an Integrated Microfluidic Device, Anal. Chem. 2001, 73, 565-570.

8. Rasooly, A. and Jacobson, J. (2006) Development of biosensors for cancer clinical testing. Biosensors and Bioelectronics 21, 1851-1858.

9. Lazcka, O., Del Campo, F. J. and Munoz, F. X. (2007) Pathogen detection: A perspective of traditional methods and biosensors. Biosensors & Bioelectronics 22, 1205-1217.

10. Shastry, B. S. (2006) Pharmacogenetics and the concept of individualized medicine. Pharmacogenomics Journal 6, 16-21.

11. Dvorak, Z., Pascussi, J.-M. and Modriansky, M. (2003) Approaches to messenger RNA detection: Comparison of methods. Biomedical Papers (Olomouc) 147, 131-135.

12. Pohl, G, Shih, I-M. (2004) Principle and applications of digital PCR. Expert Rev Mol Diagn 4, 41-47.

13. Bardea, A. and Naaman, R. (2009) Magnetolithography: From Bottom-Up Route to High Throughput. Small 5, 316-319.

14. A. Bardea, A. Baram, A. Tatikonda, R. Naaman, “Magnetolithographic patterning of inner walls of a tube: A new dimension in micro fluidics and sequential microreactors”, J. Am. Chem. Soc. 131, 18260-18262 (2009).

15. Ziv, C., Gorovits, R. and Yarden, O. (2007) Carbon Source affects PKA-dependent polarity of Neurospora crassa in a CRE-1-dependent and independent manner. Fung. Genet. Biol. 45, 103-116.

16. Borkovich, K. A. et al. (2004) Lessons from the genome sequence of Neurospora crassa: Tracing the path from genomic blueprint to multicellular organism. Microbiology and Molecular Biology Reviews 68, 1-108.

### BACKGROUND OF THE INVENTION

DNA is a linear or circular double-stranded helical polymer, each strand contains a sugar-phosphate backbone composed of deoxyribose sugar moieties substituted by phosphate groups at their 5′ and 3′ hydroxyls, the sugar groups being connected to 4 purine and pyrimidine bases usually indicated by their initial letters, namely, adenine (A), guanine (G), cytosine (C) and thymine (T). The two single strands of the DNA (ssDNA) are connected through hydrogen bonds between the complementary base pairs A-T and C-G (the Watson-Crick base pairs), of the two strands.

Genes are segments of DNA molecules containing all the information required for synthesis of a product, namely, a protein/polypeptide chain with a certain biological function or an RNA molecule. A mutation in a gene, e.g., a change in one or more base pairs of the normal gene, may result in a protein product with a change in biological function and, thus, in a genetic defect or disease.

In the biological process of synthesis of polypeptide chains, the first step is transcription whereby the double-stranded DNA (dsDNA) serves as a template for synthesis of a single-stranded ribonucleic acid (RNA) with a base sequence complementary to one strand of the double-stranded DNA. In the second step, the translation, the polypeptide chain is synthesized using the RNA as a template. The amino acid sequence of the protein is completely determined by the sequence of bases in the RNA, which in turn is determined by the sequence of bases in the DNA of the gene from which it was transcribed. To date, there are several methods of DNA and RNA detection and identifications that are based on several basic technologies like DNA chips [1, 2], electrochemical-based detection [3], nanopores [4], and CMOS based devices [5]. For many medical, forensic, environmental and biological research applications, sensitive detection of a single oligonucleotide, either DNA or RNA, can provide a significant advance. Methods for analysis of gene expression and for multi species DNA/RNA screening are also of high demand.

Polymerase chain reaction (PCR)-based techniques have been instrumental in addressing many needs involving nucleic acid detection [6]. A single DNA molecule detection has been demonstrated using polymerase chain reaction (PCR) [7]. Since sensitivity is a critical issue, generally the methods described above are based on the amplification of the number of DNA molecules prior to their analysis. The amplification of the number of DNA molecules is performed either by using a PCR method or by allowing the organisms, from which the sample is taken, to multiply.

RNA also becomes an important diagnostic tool. However, its detection is even more challenging than DNA detection. The reverse transcription polymerase chain reaction (RT-PCR) is the most sensitive method for the detection of low-abundance mRNA, often obtained from limited tissue samples. The importance of sensitive RNA and mRNA detection is constantly increasing in medicine, for advancing the diagnostics and treatment of cancer, myocardial infarction, and diseases caused by bacteria and viruses [8-9]. It is an essential tool for utilization of personalized medicine [10]. These applications have stimulated the development of various methods for DNA and RNA detection [11-12]. However, the PCR method is a complex technique, requires expertise, and takes a relatively long time to perform, which also contributes to the costs involved [6]. There are substantial problems associated with its true sensitivity, reproducibility and specificity and, as a quantitative method, it suffers from the problems inherent in PCR. The recent introduction of fluorescence-based kinetic RT-PCR procedures significantly simplifies the process of producing reproducible quantification of mRNAs and promises to overcome these limitations. Nevertheless, their successful application depends on a clear understanding of the practical problems, and careful experimental design, application and validation remain essential for accurate quantitative measurements of transcription.

Despite the significant advances in DNA detection methods, there is still a growing need for simple new technologies that exhibit comparable or improved sensitivity.

### General Description

As described above, detection today of DNA and RNA requires amplification processes. This requirement introduces several difficulties. In the case of using PCR, it is difficult to make it a quantitative method [6]. Namely, it is still a challenge to keep good correlation between the amount of DNA obtained after the amplification to the original amount. This is especially true when the original amount is very small and the sample is not clean. Organism multiplication takes time and delays the time between sampling and sample identification. By using both amplification methods, the DNA/RNA detection and identification cannot be performed in place where the sampling occurs and requires highly professional laboratories.

Hence, there is a need for a sensitive, inexpensive, rapid and mobile tool for detecting and analyzing and identifying analytes such as foreign material, toxin or DNA and RNA, since their detection can be the key for a sensitive detection and identification of viruses and bacteria. Moreover, there is a need for a system that can detect and identify both DNA and RNA simultaneously. In addition, DNA becomes an important tool in forensic science, where its detection and identification becomes a revolutionary tool. Moreover, there is a special need for a detection and identification method that can be performed rapidly in” the field” (in place where the sampling occurs) with no need to transport and deliver the sample to professional laboratories.

The present invention provides a method and sensor for detection and identification of analytes such as foreign material or DNA and RNA in particular in low concentration. The method can be used for diagnostic as well as for forensic applications. The present invention provides a new technique for the detection of analytes such as DNA/RNA which does not require the use of any DNA or RNA amplification, e.g. without the use of PCR. The sensitivity of the method allows the detection of few molecules (down to a single one) independent on the concentration of the sample. The technique is simple to use and can be applied “in the field” by a relatively simple set-up.

Therefore, there is provided a sensor for analyte(s) detection in a fluid sample. The sensor comprises at least one tube-like member carrying an arrangement of probes patterned thereinside.

In some embodiments, the present invention provides DNA and RNA molecule detection and identification using a patterned capillary tube (PCT). In this connection, it should be understood that since the ratio between the area of the inner wall surface and the volume is high, the probability of analyte molecules to interact faster with molecules probe that is bound to the inner surface of the tube is high. Hence, the smaller is the diameter of the tube, the higher is the probability of the analyte to react with the probe.

In this connection, it should be noted that in the context of the present invention, the tube-like member is not limited to a cylindrical geometry, neither linear member nor circular cross-section, but refers to a closed-loop inner surface of the sensor device.

The sensor is able to detect and identify various analytes contained in the fluid sample while the sample flows through the tube-like member. The arrangement of probes defines identifiable positions along the tube-like member. Each probe contains a different substances such that a surface of each probe is configured and operable to react with a specific analyte contained in the fluid sample while flowing through the cavity to enable a surface modification, thereby enabling selective and specific quantitative detection of the specific analyte in the sample by identifying one or more of the locations of interaction between the respective one or more probes and the analyte. The tube-like member defines an inner cavity for flowing a fluid sample therethrough and comprises an inner surface at least partially patterned. The pattern is in the form of an array of spaced-apart locations comprising locations of interaction carrying different probes immobilized to the inner surface of the tube-like member.

The interaction between the surface of a probe and a specific analyte may induce chemical and/or physical binding between them.

The probe may comprise different substances including biological moieties, chemical moieties or other binding molecules having the capability of binding with foreign materials of the sample (for example water and food contaminating species).

The probe may comprise different biological moieties such as DNA sequence, RNA sequences, different antibodies or antigens, proteins or full cells, etc. The biological moiety may be for example a peptide based molecule (peptide, polypeptide or protein, glycoprotein, etc.) or a nucleic acid based moiety (e.g. an oligonucleotide or polynucleotide) or any modification thereof. The interaction between the biological moiety and the specific analyte contained in the fluid sample may include protein/protein, nucleic acid/protein or nucleic acid/nucleic acid interaction. Some non-limiting examples of such binding moieties include enzyme/substrate, antigen/antibody, ligand/receptor, nucleic acid sequence/nucleic acid sequence, nucleic acid sequence/nucleic acid binding proteins, sugar/lectin, enzyme/inhibitor, enzyme/co-factor etc.

The interaction between the biological moiety and the specific analyte contained in the fluid sample may include the specific binding between complementary strands of nucleic acids sequences causing natural hybridization between them. Each of the bound nucleic acid sequences may be a sequence composed of DNA nucleotides, RNA nucleotides or a combination of both types, and may include natural nucleotides, chemically modified nucleotides and synthetic nucleotides. Accordingly, binding pairs would include DNA-DNA interactions, DNA-RNA interactions, RNA-RNA interactions, etc. Whilst the following examples depict use of nucleic acid sequences as linking biological moieties, the present invention is not limited thereto, as a person skilled in the art of the invention would easily modify the procedure to use other biological moieties (such as proteins).

As indicated above, such a nucleic acid binding is the interaction between complementary strands of nucleic acids sequences; each of the bound nucleic acid sequences may be a sequence composed of DNA nucleotides, RNA nucleotides or a combination of both types and may includes natural nucleotides, chemically modified nucleotides and synthetic nucleotides.

In this connection, it should be understood that the tube-like member configuration has clear advantages over a DNA chip configuration.

In some embodiments, the probe extends along a circumferential region at the corresponding location on the inner surface of the tube-like member. The area covered with the probe at the inner surface of the tube-like member may have a ring-like geometry.

In the present invention, the inner surface of a tube-like member is patterned by an arrangement of DNA-based probes (e.g. rings). The new detection and identification method of the present invention is based on patterning the inside of a tube-like member with probe molecules at well defined locations and flowing a solution containing the analyte oligomers through the tube-like member.

Alternatively, the tube-like member may be patterned by conventional patterning techniques on a flat/planar flexible substrate. The patterned planar flexible substrate is then rolled to form a tube-like member.

The sensitivity of the sensor of the present invention is significantly greater that the sensitivity two dimensional DNA/RNA chip. The sensitivity of the sensor is determined by a number of molecules of a specific analyte in the sample while being independent of concentration of the analyte in the sample. In other words, the sensor of the present invention is sensitive to the number of analyte molecules and not to their concentration, as the case is in two-dimensional arrays. In principle, in two-dimensional array, the concentration of the analytes has to increase with the number of pixels and with the area of the chip. This is required in order that the probe molecules, on each pixel, will have a probability for reacting with at least a single analyte.

In the present invention, since the solution containing the analytes is flowing through the tube, each molecule has a probability to hit the wall of the tube and to interact with the probe molecules wherever they are located on the inner surfaces of the tube. Having a large number of probe molecules at the inner surface of the tube (being adsorbed), induces the detection of a single analyte molecule or species, like a cell, of interest. The sensitivity is affected by the size of the surface of interaction and the probability of the analyte species to collide with this area.

In some embodiments, there is provided a novel ultra-sensitive, fast, and mobile method of detection and identification of both DNA and RNA. The novel method is highly sensitive down to a single molecule level, independent of the concentration of the oligonucleotide in the sample. It is based on patterning the inner surface of a tube (e.g. capillary glass tube) with probe DNA molecules (e.g. rings of different single-stranded DNA probes), subsequent exposure to the analyte by flowing a sample (e.g. a solution containing the analyte oligomers) through the tube, and detecting the sample-analyte hybrid on the basis of a fluorescence signal.

For example, a DNA polymerase may be used for detecting DNA, and reverse transcriptase may be used for detecting RNA. The signal may be obtained with deoxynucleoside triphosphate (dNTP) labeled with a fluorophore. The sensor allows the detection of the analyte in serum without any pretreatment, and can be used to simultaneously detect various DNA and RNA analytes.

The present invention provides a novel detection and identification method and system for DNA and RNA.

In some embodiments, the sensor is configured with a predetermined controllable sensitivity defined by selecting of at least one of the following parameters: a geometrical parameter of the tube-like member, a dimension of a feature of the pattern, and a parameter of the fluid sample flowing through the tube-like member. At least one parameter of the fluid sample flowing through the tube-like member comprises at least one of the following: a ratio between a diffusion rate of the sample, a flow rate of the sample through the tube-like member, and one or more predefined conditions of the fluid sample for hybridization.

The solution can flow through the tube-like member back and forth. The probability for an analyte to hit the wall in the tube depends on the diameter of the tube, as smaller is the diameter so larger is the probability to hit the wall. The number of times that the DNA molecule hits the adsorbed probe on the surface (N) depends on the diameter of the tube (d), the length of the area covered with the probe (L), and the ratio between its diffusion rate (R_{D}) and the flow rate (F) through the tube.

Hence: N=LR_{D}/dF (1)

If the reaction probability between the analyte and the probe is given by p, then in order to detect a single analyte:

pN≧1 (2)

Namely as longer is the probe adsorption ring and as faster is the diffusion to the wall so higher is N. Small diameter and slow flow rate increases also N.

The ability to control the probability of the analyte to react with the probe, as shown in Eq. 1, is a feature of the present invention eliminating the need for DNA or RNA amplification. Therefore, the diameter of the tube, the length of the area covered with the probe (L), and the ratio between its diffusion rate (R_{D}) and the flow rate (F) through the tube are selected to control the sensitivity of the sensor. The sensor of the present invention may comprise a control unit configured and operable to adjust parameters of the fluid sample such as the flow rate through the tube and the tube's temperature for a given geometry (e.g. cross-sectional dimension) of the tube. The geometrical parameters of the tube (e.g. the diameter of the tube, the length of the area covered with the probe (L)) are selected to provide certain sensitivity.

There is also provided a sensing system for use in detection of one or more analytes in a fluid sample. The system comprising a sensor comprising at least one tube-like member defining an inner cavity for flowing a fluid sample therethrough and comprising an inner surface at least partially patterned. The pattern is in the form of an array of spaced-apart locations comprising locations carrying different probes immobilized to the inner surface of the tube-like member. The different probes comprises different biological moieties such that each probe is configured and operable to react with a specific analyte in a sample while flowing through the cavity creating a probe-analyte bond, identifiable by generation of a radiation response. The system comprises a control unit comprising a detector for detecting the radiation response. The control unit is operable for analyzing the detected response to determine one or more of the probe locations where the response have been originated as a result of interaction between the respective one or more probes and the analyte, thereby identifying a specific analyte in the sample.

In some embodiments, the control unit comprises at least one conditional sensor. Each conditional sensor is configured and operable for controlling at least one parameter of the fluid sample.

In some embodiments, the sensor comprises a sensor configured and operable for controlling a flow rate of the fluid sample and/or a temperature sensor configured and operable for controlling the temperature of the fluid sample and/or a pH sensor configured and operable for controlling the pH of the fluid sample.

There is also provided a method for use in detection of one or more analytes contained in a fluid sample. The method comprises flowing the fluid sample along a flow cavity and causing successive interactions between an array of surface regions of the cavity carrying different probes at fixed locations of interaction along the cavity capable of reacting with different analytes in the sample, thereby resulting in different surface modifications of the cavity; detecting data indicative of the surface modification and selectively and specifically identifying at least one analyte molecule quantitatively in accordance with the one or more fixed locations of interaction corresponding to the surface modification.

In some embodiments, the step of detecting and identifying the analyte depends on the number of molecules of said analyte in the sample and is independent of a concentration of the analyte in the sample.

In some embodiments, the step of detecting and identifying the analyte comprises analyzing a radiation response from one or more of the probes and determining one or more of the probe locations where the response have been originated as a result of the reacting.

In some embodiments, the step of reacting with different analytes in the sample comprises hybridization between a biological moiety of the probe and the corresponding analyte.

In some embodiments, the step of detecting and identifying of the one or more analytes comprises flowing through the cavity at least one replication biological moiety marked with one or more fluorescent labels respectively.

In some embodiments, the method comprises providing desired sensitivity of the detection and identification of the analyte in the sample by controlling at least one of the following parameters: a geometrical parameter of the flow cavity, a dimension of a feature of the probes, and a parameter of the fluid sample flowing through the flow cavity.

In some embodiments, the method comprises providing the array of the surface regions of the cavity carrying different probes at fixed locations along the cavity, by patterning an inner surface of a tube-like member. The pattern is in the form of an array of spaced-apart locations comprising locations carrying different probes immobilized to the inner surface of the tube-like member. The different probes comprise sequence-specific different substances. Each probe is configured and operable to react with a specific analyte in the sample while flowing through the cavity.

In some embodiments, the step of patterning comprises immobilizing a plurality of substances corresponding to different probes respectively to spaced-apart locations along the inner surface of the tube-like member. The step of immobilizing comprising: (a) patterning the inner surface of the tube-like member, by magneto-lithography for example, to create an array of locations functionalized for binding to the different substances and spaced by regions of the inner surface incapable of any such binding; (b) flowing a first type of the substances, functionalized for binding to the locations, while applying a magnetic field pattern to the cavity such as to permit binding of the substance of the first type to one or more specific locations and prevent the binding to other of the locations; (c) repeating step (b) a predetermined number of times, each time modulating the magnetic field pattern for binding another type of the plurality of substances to another one or more locations, until binding all the substances from the plurality of substances to different locations.

In some embodiments, the step of patterning of the inner surface of the tube-like member comprises applying a first modulated magnetic field in the vicinity of the cavity thus creating a certain pattern of the plurality of locations of interaction to be obtained on the inner surface of the tube-like member; interacting the inner surface of the tube-like member with magnetic particles under the application of the first modulated magnetic field, thereby attracting the magnetic particles to the locations of interaction while being substantially not attracted to spaces between the locations of interaction, thus creating in the inner surface of the tube-like member the certain pattern of locations interacted with the magnetic particles; flowing a first reacting agent through the tube-like member thereby interacting the first reacting agent via chemical recognition and/or biological recognition with the inner surface of the tube-like member within spaces between the locations and binding the first reacting agent with the inner surface in the spaces, while the magnetic particles blocking the binding of the first reacting agent to the locations; removing at least a portion of the magnetic particles by removing an effect of the magnetic field, resulting in a negatively patterned inner surface; flowing a second reacting agent through the tube-like member thereby binding the second reacting agent via chemical recognition and/or biological recognition with the inner surface of the tube-like member at the locations free of the first reacting agent bonded to the inner surface of the tube-like member, to create the array of locations functionalized for binding to the probe substances; applying a second modulated magnetic field in the vicinity of the cavity configured to define a predetermined number of the locations to be excluded from interaction with one or more of the probe substances; and interacting the inner surface of the tube-like member with magnetic particles under the application of the second magnetic field, thereby attracting the magnetic particles to the predetermined number of locations, thus blocking the predetermined number of locations from interaction with substances.

In some embodiments, the modulated magnetic field is applied by a magnetic pattern generator defining an arrangement of ferromagnetic material at identifiable well-defined positions corresponding to well-defined locations along the tube-like member.

In some embodiments, the step of interacting of the inner surface of the tube-like member with magnetic particles comprises flowing a solution containing the magnetic particles through the cavity such that the particles are attracted towards the identifiable well-defined positions of the magnetic pattern generator.

In some embodiments, the method comprises detecting and identifying at least one DNA sequence and at least one RNA sequence simultaneously. The selective identification of said analyte may further comprise flowing a first developing solution along said flow cavity including a biological substance elongating one type of analyte and subsequently injecting another developing solution along said flow cavity containing another biological substance required for a different elongation process.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1A illustrates a schematic illustration of the sensor of the present invention;

FIG. 1B illustrates the production of the DNA patterned tube-like member;

FIGS. 1C-1D illustrate the detection of DNA and amplification of the signal by using DNA polymerase and nucleotides (dNTPs) including dCTP carrying a fluorescent label;

FIG. 1E illustrates the detection of RNA and amplification of the signal by using reverse transcriptase and dNTPs which include dCTP carrying a fluorescent label;

FIGS. 2A-2B shows control experiments, in particular, FIG. 2A illustrates the fluorescence signal obtained from each of the experiments; FIG. 2B shows a plot of the fluorescence intensity measured in each experiment;

FIGS. 3A-3B show a fluorescence signal as a function of analyte concentration obtained for a tube fully covered from the inside with probe molecules; in particular, FIG. 3A shows a fluorescence image and a plot of the fluorescence intensity as a function of the position in the tube; FIG. 3B shows a plot of total intensity signal versus the number of DNA molecules in the solution flown through the tube;

FIG. 4 show a fluorescence signal of very low amount of analyte;

FIGS. 5A-5D show a fluorescence signal as obtained when the tubes was modified with DNA₁ probe and DNA₂ probe on two sites in the inner surface of the tube, according to the procedure described in FIG. 1B; in particular, FIG. 5A shows a fluorescence signal from the DNA₁ and DNA₂ probe sites after only analyte 1 was injected into the tube; FIG. 5B shows a fluorescence signal from the DNA₁ probe and DNA₂ probe sites after analyte 2 was also injected into the tube, FIG. 5C shows a fluorescent signal in DNA₁ and DNA₂ probe sites after only analyte 2 was injected into the tube; FIG. 5D shows a fluorescence signal in DNA₁ probe and DNA₂ probe sites after analyte 1 was also injected into the tube;

FIG. 6A shows a fluorescence signal of the DNA analyte. The inset shows the fluorescence intensity when a constant number of molecules were detected;

FIGS. 6B-6C are the actual images of the fluorescence signal for various concentrations of DNA molecules;

FIGS. 7A-7B show a fluorescence signal of the sensor of the present invention when a small number of DNA molecules were passed through the tube;

FIG. 8A shows a concentration-dependent curve of the RNA analyte;

FIG. 8B shows a time-dependent fluorescence signal of 10-11M N. crassa RNA analyte;

FIG. 9A shows a comparison between the fluorescence signal of a sensor exposed to a 1 nM RNA solution from tomato and a mixture sample of 10 pM RNA from N. crassa with 1 nM RNA from tomato;

FIG. 9B shows a concentration-dependent curve of sensing a N. crassa RNA analyte in a mixture containing human serum;

FIG. 10 shows a comparison between the sensor of the present invention and RT-PCR; and;

FIGS. 11A-11B show the specificity and selectivity of the sensor of the present invention with two different probes.

### DETAILED DESCRIPTION OF EMBODIMENTS

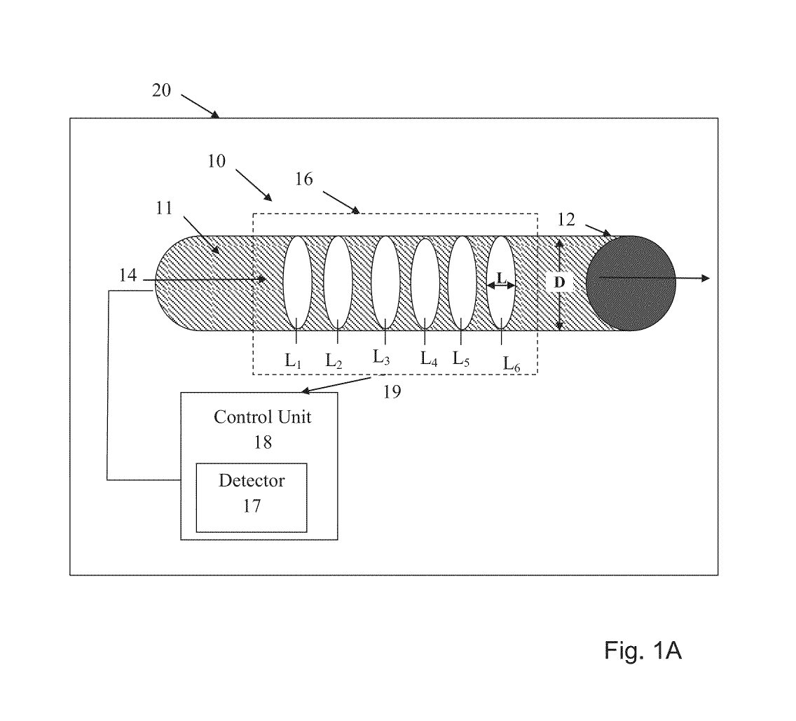

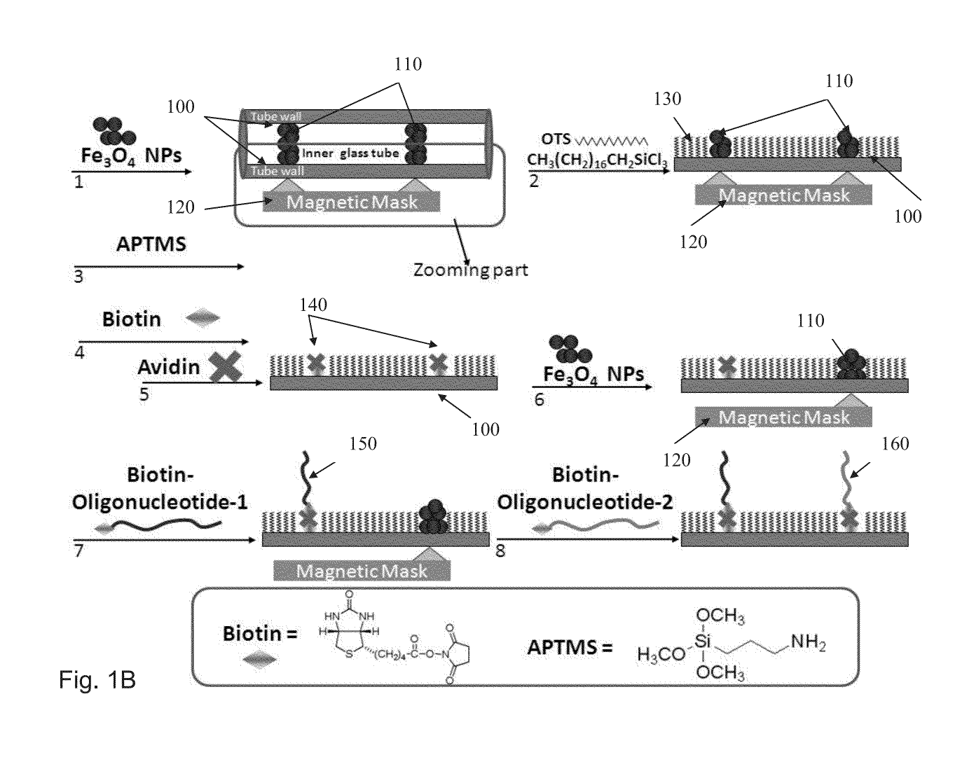

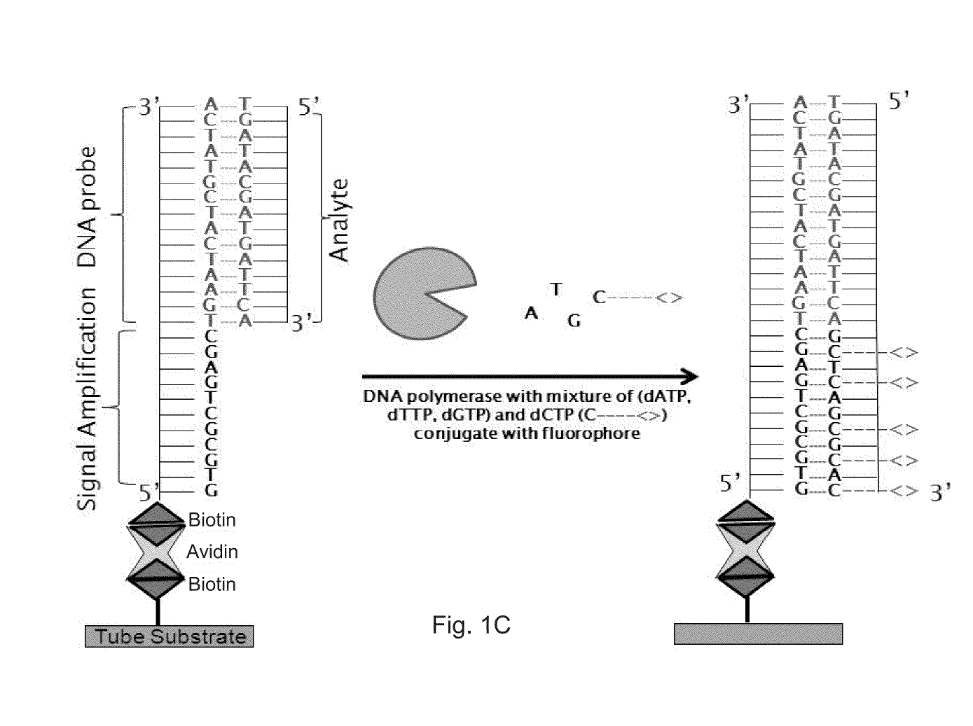

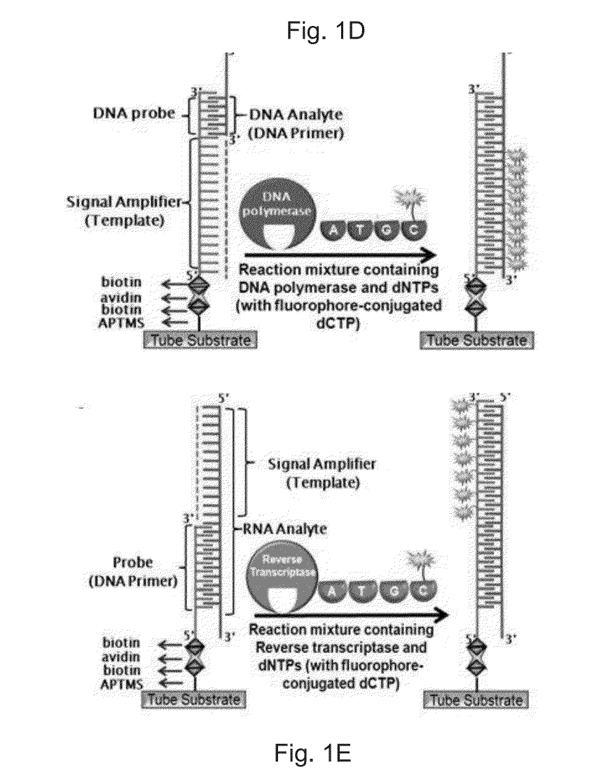

Reference is made to FIG. 1A schematically illustrating a sensing system for use in detection of one or more analytes in a fluid sample of the present invention. The system 20 comprises a sensor 10 comprising a tube-like member 12 defining an inner cavity 11 for flowing a fluid sample 14 therethrough and a control unit 18 comprising a detector 17 for detecting radiation response 19 from the sensor 10.

The tube-like member 12 comprises an inner surface at least partially patterned. The pattern is in the form of an array of spaced-apart locations comprising locations of interaction L₁-L₆ carrying different probes 16 immobilized to the inner surface of the tube-like member 12. The probes 16 comprise different substances. Each probe is configured and operable to react with a specific analyte in the sample 14 to create a hybridized probe-analyte while flowing through the cavity 11 thereby enabling detection of the specific analyte in the sample 14 by identifying one or more of the locations of interaction L₁-L₆ between the respective one or more probes 16 and the analyte.

The control unit 18 is operable for analyzing the detected response (e.g. signal from fluorescent sensors) to determine one or more of the probe locations where the response have been originated as a result of interaction between the respective one or more probes and the analyte, thereby identifying a specific analyte in the sample 14.

In some embodiments, chromophore molecules are added to the hybridized probe-analyte and can be detected by fluorescence. In this case, the control unit 18 comprises an excitation source (LED, laser etc., which is not shown) for exciting the different probes 16 (i.e. chromophore molecules interacted with the probe-analyte) to thereby induce generation of a radiation response (fluorescence) from the sensor 10. The control unit 18 may also provide power activating the excitation source.

In some embodiments, the control unit 18 comprises at least one of environmental/conditional sensor (not shown). Each such sensor is configured and operable for controlling at least one parameter/condition of the fluid sample 14 and/or the sample flow environment. The sensor may be configured and operable for controlling a flow rate of the fluid sample 14 and/or may be a temperature/pH sensor configured and operable for controlling the temperature of the fluid sample 14. The selectivity of the hybridization is affected by the conditions in which this process is done, i.e., the solvent (ionic strength) and the temperature. A person skilled in the art of the invention would be able to provide the necessary conditions (pH, temperature, etc) that would be necessary to enable the binding to take place at sufficient specificity to reduce (preferably minimize) non-specific interactions.

In this connection, it should be noted that the sensor of the present invention provides a novel configuration. Such sensor may be a stand-alone device or may be mounted with any control unit of any type if needed including the configuration of the present invention.

In some embodiments, the analyte sensor device comprises an arrangement of a plurality of tube members, all together creating a long one- or two-dimensional sensing system capable of detecting hundreds of different sequences of DNA/RNA. Each member may detect a specific sequence of DNA/RNA. The arrangement of tube members may be formed by connecting a plurality of tube's members together along one or two axes (e.g. serpentine-like or spiral-like configuration), the fluid sample successively passing through each member. Some of the tubes may be made of flexible material enabling the connection between the different tubes or the tubes may be connected via flexible connections.

In some embodiments, the different probes 16 comprise different biological moieties such as DNA/RNA sequences. The tube-like member 12 thus carries an arrangement of DNA/RNA-based probes 16 patterned thereinside. The arrangement of DNA/RNA-based probes 16 maps the tube-like member 12 at identifiable positions (locations of interaction) L₁-L₆ along the tube-like member 12. Each probe contains different DNA sequence such that a surface of each probe is configured and operable to react with a specific analyte contained in the fluid sample 14 to enable a surface modification. The sensor 10 is thus able to detect and identify various analytes contained in the fluid sample 14 while the sample 14 flows through the tube-like member 12.

In this specific and non-limiting example, each probe defines a ring-like shape.

In some embodiments, the control unit 18 is configured and operable to select at least one parameter of the fluid sample through the tube-like member to control a sensitivity of the sensor 10 for a given geometry of the tube.

The geometrical parameters of the tube-like member such as a diameter D of the tube-like member 12, a feature dimension of the pattern comprises a length L of an area covered by the probes are selected to provide certain sensitivity.

Reference is made to FIG. 1B showing schematically an example of a process of creation of an arrangement of DNA/RNA-based probes patterned inside a tube-like member/flow cavity and illustrates a scheme of a surface modification process. The patterning of the inside of the tube may be performed by applying the magneto-lithography method [13-14].

In some embodiments, the array of the surface regions of the cavity carrying different probes at fixed locations along the cavity is created by patterning the inner surface of the tube-like member. The pattern is in the form of an array of spaced-apart locations comprising locations carrying different probes immobilized to the inner surface of the tube-like member and comprising different substances.

In this specific and non-limiting example, the probe molecules are patterned on the inner surface of a 15-mm-long, 300-μm inner diameter glass tube. The patterning is achieved through an 8-steps procedure in which the tube is placed in a constant magnetic field with rings of ferromagnetic metals where the probe rings are to be adsorbed.

As illustrated in the figure, in step 1, the internal/inner surfaces 100 of the tube are first covered with magnetic particles 110 (e.g. ferromagnetic nanoparticles (NPs)) by applying a first modulated magnetic field in the vicinity of the tube-like member/cavity. The modulated magnetic field may be applied by a magnetic pattern generator 120 (denoted as a magnetic mask) defining an arrangement of ferromagnetic material at identifiable well-defined positions corresponding to well defined locations along the tube-like member. The magnetic pattern generator 120 is configured and operable to modulate a magnetic field to induce varying magnetic properties to a magnetic field according to a desired pattern and applying a modulated magnetic field.

The magnetic pattern generator may include a physical element or elements accommodated between a magnetic field source and a substrate to be patterned; or may be constituted by operation of a magnetic field source to electronically affect the magnetic field properties (profile). Thus, in some embodiments, the magnetic pattern generator is a mask placed between a constant magnet (magnetic field source) producing the patterned magnetic field, and the substrate, the mask being located either a topside or backside of the substrate or is spaced-apart from the backside of the substrate.

The magnetic pattern generator 120 is placed at the regions where the rings of DNA probe are supposed to be form. In this specific and non-limiting example, the magnetic field generator 120 defines two sites along the tube. A solution containing the nanoparticles (NPs) 110 (e.g. a solution containing 10-nm diameter magnetite Fe₃O₄ super paramagnetic nanoparticles (NPs) is flown (e.g. is injected) through the tube. The NPs are assembled at the position of the magnetic pattern generator 120 (e.g. next to the rings or tips of ferromagnetic metals), inside the tube, mask these positions and protect these positions from adsorption.

The Fe₃O₄ particles are stable colloidal dispersions of single domain magnetic particles, 10 nm in diameter. The particles are held in suspension by dispersing agents like oleic acid, which are compatible with both the carrier fluid and the magnetic particle. The magnetic nanoparticles were used after they immersed in ethanol, 0.03% volume.

Then, in step 2, a first reacting agent 130 is flown through the tube to interact with the substrate in the spaces between the selected regions of interaction between the magnetic particles and the substrate to create a negative patterning on the surface of the tube. The first reacting agent 130 is selected to react with substrate (e.g. glass tube) and to be inert to any of the sampling materials (the analyte), blocking the regions between the selected regions of interaction between the magnetic particles and the substrate from reacting with any substance. In this specific and non-limiting example the first reacting agent is made from octadecyltrichlorosilane (OTS) molecules. The first reacting agent may also be made from other chlorosilane alkyl chain/siloxane alkyl chain. The solution of OTS is flown (e.g. is injected) through the tube and the entire internal/inner surfaces 100 of the tube are coated with the first reacting agent 130 (e.g. OTS monolayers), except at the places previously masked by the NPs 110 and already protected by the NPs 110. The surface covered (locations of interaction) with the NPs 110 is protected and therefore does not react with the OTS.

In the step 3, the magnetic pattern generator 120 is removed in all of the locations of interaction, and all the particles 110 are washed off. The exposed areas, not covered by the particles, can be covered by molecules that chemically bind to the substrate and can further bind other molecules. The patterned regions (e.g. the two exposed sites that were not protected by NPs) in the inner surface of the tube are functionalized by amino propyle trimethoxy silane (APTMS) to further react with a second reacting agent (i.e. biotin). The exposed surface is thus coated with amine propyl 3-methoxysilane (APTMS) via a covalent link to the glass surface.

Alternatively, the magnetic pattern generator may be configured and operable to remove the magnetic field from only one location while keeping it in all the others. The particles are then “washed” leaving only one specific location of interaction exposed while other locations are still covered by the nanoparticles which are kept there by the magnetic field. This specific location will serve at immobilizing a different type of biological moieties later on. This step will then be repeated location-by-location to immobilize multiple different types of biological moieties respectively.

In step 4, the patterned regions (surface exposed) were coated with a second reacting agent 140 (e.g. biotin or by chemically functionalized single stranded DNA) operable to further immobilize DNA-based probes to the inner surfaces 100 of the tube.

Thus, according to the two different embodiments, the second reacting agent may be of the same type in all the locations or may be different for different locations.

The second reacting agent is selected to be capable of chemical binding with the substrate and with many different biological moieties-based probes, or may be selected for specific DNA probe only. The second reacting agent may cross link between the inner surface of the tube and the probe by tailoring a functional group that binds to the inner surface of the tube and at least one other functional group that binds to the probe. The second reacting agent is thus selected to have at least one functional group having a chemical affinity with the inner surface of the tube, for binding the reacting agent molecules to the inner surface of the tube and at least one another functional group of each molecule of the second reacting agent is prepared for reacting selectively with a corresponding one of the probe.

In this specific and non-limiting example, the patterned regions are exposed to a solution of biotin N-hydroxy-succinimide-ester (NHS-biotin) operable as an immobilizing reacting agent 140 reacting with the functional patterned surface (amino functional) functionalized in step 3. The biotin NHS is bound to the amine functional group through amid bond between biotin and APTMS, patterning the inner tube surface with biotin.

In step 5, the inner surface of the tube is exposed to a solution of avidin for creating biotin-avidin complexes based on its bio-affinity at two biotin patterned sites.

In some embodiments, for adsorbing different DNA/RNA probes at each ring, the ferromagnetic rings are repositioned in the same places as before but one location is left empty.

In step 6, the magnetic pattern generator 120 is repositioned at a single well defined position. The magnetic field is therefore applied at one site of a biotin-avidin complex and a solution containing the nanoparticles 110 is flown/injected through the tube again to mask this site. The particles 110 are assembled at the position defined by the magnetic pattern generator 120 (e.g. the ferromagnetic tips) and protect this position from reaction/interaction with the probe in the next step.

One type of DNA probe 150, modified by biotin (e.g. a solution of Oligonucleotide-1 modified with biotin), is flown/injected in step 7 through the tube and binds to the non-protected biotin-avidin region.

The same procedure is repeated for each ring, with different DNA probes.

In step 8, the magnetic pattern generator 120 is removed, the particles 110 are washed and therefore one of the avidin sites is deprotected. Next, another type of DNA probe 160 (e.g. solution of Oligonucleotide-2 modified with biotin) is flown/injected through the tube and interact with the deprotected avidin site.

At the end of this process, the tube is patterned with several rings, each comprising a different (sequence-specific) probe.

In this specific and non-limiting example, this process results in two sites along the tube modified with different probes (e.g. sequences of oligonucleotide).

In this connection, it should be noted that this process can be made automatically or semi-automatically by using switchable magnetic generators preprogrammed for successively creating different magnetic field patterns thus enabling the patterning of an array of rings containing each different DNA probe on the inside surface of the tube. The switchable magnetic generators are configured and operable to switch the magnetic field on and off to control the magnetic pattern, patterning different biological moieties-based probes at different locations. Therefore, an arrangement of biological moieties-based probes defining identifiable positions along the tube-like member is created. Each probe contains different biological moieties such that a surface of each probe is configured and operable to react with a specific analyte contained in the fluid sample to enable a surface modification.

The following describes in details a specific and non-limiting experiment performed by using the teachings of the present invention. A constant magnetic field was applied from underneath the tube. 300 μm inner diameter glass tubes were located 0.5 cm away from the magnet after they were clean by boil ethanol and then by 2% of hydrofluoric acid. Between the magnet and the glass tube an iron mask was positioned. The iron mask was composed from two spaced-apart tips located at 1 mm from each other, each having a width of 50 μm. The magnetic field applied to the tips was of about 100 Gauss. A solution containing 1 mg ml⁻¹ of the Fe₃O₄ NPs in ethanol was injected into the tube. The NPs were assembled according to the gradient of the magnetic field within the tube, namely near the sites of the tips. They protect these sites from chemical modification. A 1 mM solution of OTS (octadecyltrichlorosilane (90+%, Aldrich)) in dry toluene was injected to the tube for 30 sec at room temperature then the tube was washed with toluene and ethanol and the NPs were removed. A 1 mM solution of amino propyle trimethoxy silane (APTMS) in ethanol was injected to the tube and incubated for two hours at room temperature, then the tube was washed with ethanol and distilled water. As a result of this process, two patterned regions (sites) are formed within the tube, patterned with the aminosilane, one for the detecting DNA and the other for RNA.

A solution of 1 mg ml⁻¹ N-hydroxy-succinimide-biotin (NHS-biotin) was injected to the tube and reacted with the amino functional patterned surface for one hour at room temperature. Following this process, the previously amine coated sites are now coated with biotin. A mixture of 10 μM avidin (streptavidin) with 100 nM bovine albumin was immersed into 50 mM buffer phosphate (pH 7.5) and injected to the tube. The tube was incubated during one hour at room temperature and then washed by buffer phosphate (BP). The result of the bio-recognition process between biotin and avidin is the binding of avidin to the biotin sites along the inner surface of the tube.

Reference is made to FIGS. 1C-1D showing examples of the technique of detection of DNA and amplification of the signal by using DNA polymerase and nucleotides including dCTP carrying a fluorescent label e.g. modified by dye molecules.

The solution containing the oligonucleotide analyte, which complements the sequence of the surface-bound DNA/RNA probes, is flown through the tube, followed by a “developing” solution containing the enzyme DNA polymerase or reverse transcriptase (RT) for detecting the DNA or RNA, respectively, and various nucleotides marked with dye molecules e.g. a deoxynucleoside triphosphate (dNTP) mixture labeled with fluorophores. The DNA polymerase requires a primer for starting the replication process. The DNA probe is used here as a template and the analyte is used as a primer. Hence, only once the analyte is bound to the probe, and only after that occurs, the nucleotides from the solution are added to the primer, namely to the analyte. In this specific example, the double-stranded nucleotide chain serve as a priming site for 5′ to 3′ extension, incorporating fluorescent nucleotides from the reaction solution. Since all the C bases that are added by the polymerase are conjugated to a chromophore, each recognition event is now coinciding with 7 chromophore molecules that were added to the hybridized probe-analyte and can be detected by fluorescence.

In this specific and non-limiting example illustrated by FIG. 1C, for immobilizing the DNA probe to the inner surface of the tube, oligonucleotides containing 51 bases and modified with biotin on 5′end were used. In principle, the DNA probe can be of any length, but practically it will contain more than 15 bases.

The DNA probe sequence (51 bases) is divided to two parts: the recognition part contains 27 bases complementary to analyte oligonucleotide and the signal amplification part contains 24 bases enriched with guanine (total of 7 bases).

The function of the recognition part is to hybridize with an analyte and the function of the signal amplification part is to amplify the signal after the hybridization has occurred. The amplification of the signal, in this specific and not limiting example, is based on polymerization of the analyte is obtained by DNA polymerase and nucleotides. The mixture of the nucleotides contains deoxy cytidine triphosphate (dCTP) labeled with fluorophore incorporated to the sequence according to the guanine bases contained in the signal amplification part of the DNA probe. The oligonucleotide analyte DNA was dissolved in 50 mM of BP (pH 7.5) for producing 1 μM of DNA probe solution. Next, the DNA probe solution was injected into the tube and incubated during one hour at room temperature. After the immobilization of the DNA probe, the tube was washed with a BP solution.

In these specific calibration experiments, the analyte DNA was dissolved in 0.1 mM BP (pH 7.5) in different concentrations. The analyte oligonucleotide contains 27 bases that are complementary to the sequence of 3′ end of the DNA probe (recognition part). Hybridization between DNA probe and the analyte was performed after the injection of 1 μl analyte solution to the tube which was modified by a DNA probe sequence. The tube was incubated for one hour at 50° C. and then the tube washed with a BP solution.

For example, for sensing the hybridization and for signal amplification, a mixture of BP solution was used. The mixture of BP contains 10 units/ml DNA polymerase enzyme (e.g. DNA polymerase I, Klenow Fragment enzyme) with 10 μM of dNTP (without dCTP), and 50 μM of dCTP labeled with fluorophore (ATTO 495). 1 μl of this mixture was injected to the tube and was incubated for 90 minutes at 42° C. Subsequently, the tube was washed with BP and dried with a stream of nitrogen. The fluorescent emission signal at a wavelength of 527 nm was measured by a fluorescent microscope (Olympus BX62 & BX50WI).

Following appropriate wash, the resulting chromophore-containing chain can be detected and quantitatively analyzed by fluorimetric methods.

In the example of FIG. 1D, to immobilize the DNA probe to the inner surface of the tube an 80-bases oligonucleotide modified with biotin at the 5′ end was used. The DNA probe was designed to include two regions: A 20-base segment that is complementary to the analyte oligonucleotide at the 3′ end and a 60-base region enriched with guanine residues (a total of 15 bases) at the 5′ end. The sensitivity of the sensor of the present invention for DNA detection was determined by using an 80-base-long probe that has two characteristics: (i) at the 3′ end, a 20-base sequence complementary to the analyte and (ii) a 60-base-long segment used for signal amplification. The latter section includes 15 guanine bases that complement the 5-propargylamino-dCTP in the developing solution. Hence, 15 chromophores were attached to each probe-analyte complex.

The function of the 3′ region was to provide a hybridization anchor for the analyte, whereas the function of the 5′ region was to provide a template for signal amplification by the appropriate polymerase. The nucleotides mixtures used contained dCTP labeled with a fluorophore to facilitate detection. These nucleotides are incorporated to the sequence according to complementary guanine bases of the DNA probe. For fixation of the probe, the oligonucleotide was dissolved in 50 mM BP (pH 7.5) to produce a 100 μM DNA probe solution. Next, the DNA (modified with biotin) probe solution was injected into the tube and incubated for 1 hour at room temperature. BP solution was used to wash DNA that did not react with the avidin.

Reference is made to FIG. 1E showing an example of the technique of detection RNA. A 40-base-long RNA oligonucleotide was synthesized and used as analyte. This analyte was detected due to the presence of two sections that include a 3′ end region of 20 bases, complementing the probe sequence and an additional section, which is used as a template for elongation. This second section included six guanine bases, designed to incorporate six cytosine fluorescent bases into the probe strand during RT polymerization. Hence, six fluorescing chromophore units were bound in every analyte-probe hybridization event.

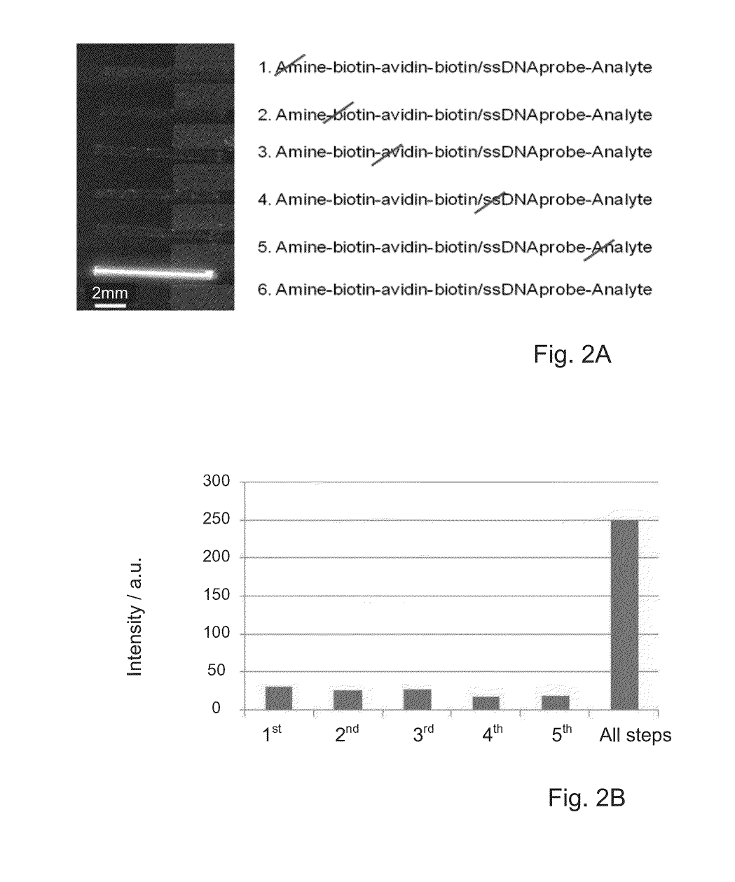

In the experiments made by the inventors, the magnetite nanoparticles (Fe₃O₄) were purchased from Liquids Research. The Fe₃O₄ particles were stable colloidal dispersions of single domain magnetic particles, 10 nm in diameter. The particles are held in suspension by dispersing agents like oleic acid, which are compatible with both the carrier fluid and the magnetic particles. The NPs were used after they were immersed in ethanol, 0.03% volume. OTS, APTMS, biotin-NHS, streptavidin from streptomyces and human blood serum were purchased from Sigma/Aldrich. Oligonucleotides were synthesized and modified by Eurofins. All oligonucleotides were purified by HPLC. DNA polymerase (I, Klenow Fragment) and Reverse Transcriptase (SuperScript II) were purchased from Bio Lab and Invitrogen, respectively. Deoxy adenosine triphosphate (dATP), deoxy thymidine triphosphate (dTTP), deoxy guanosine triphosphate (dGTP), and 5-Propargylamino deoxy cytosine triphosphate-acridine orange dye (dCTP-ATTO-495) were purchased from Jena Bioscience. The DNA and RNA used were as follows: (1) DNA oligonucleotide probe: Biotin 5′ ATT GCC TGA ATG TAC GTC TGA AAG CCT GTT GAT GCC TGA ATG TAC GTC TGA AAG CCT GTT GAC GAT GGA AGG GAA AAC AG 3′ (2) DNA oligonucleotide analyte: 5′CTG TTT TCC CTT CCA TCG TC 3′ (3) RNA oligonucleotide analyte: 5′GCC CGA ACG AAG ACA GCC CGC UGU UUU CCC UUC CAU CGU C 3′. (4) Total RNA was extracted from Neurospora crassa as described by Ziv et. al [15]. (5) Total RNA was extracted from tomato (Lycopersicon esculentum). Reference is made to FIGS. 2A-2B showing control experiments. In each experiment, one ingredient was eliminated from the system, while in the last one all ingredients were included. The background is a result of stray light in the fluorescence experiment and is independent from the content of the dyes. In particular, FIG. 2A shows the fluorescence signal obtained from each of the experiments. FIG. 2B shows a plot of the fluorescence intensity measured in each experiment. A signal of about 24 arbitrary units presents the background of the light in the system.

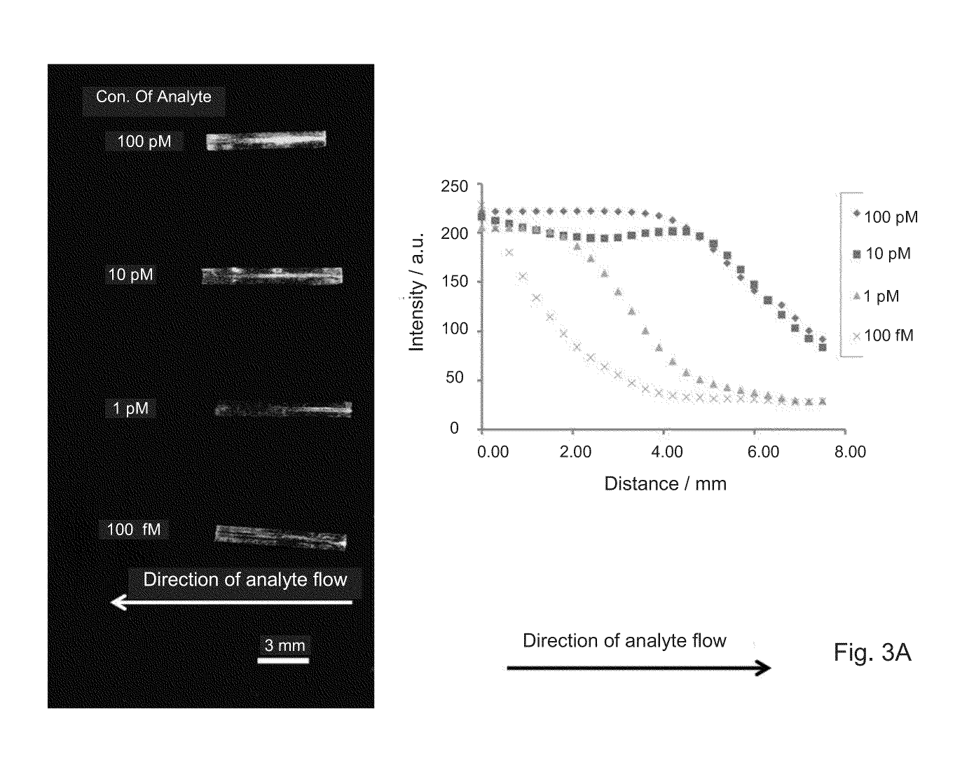

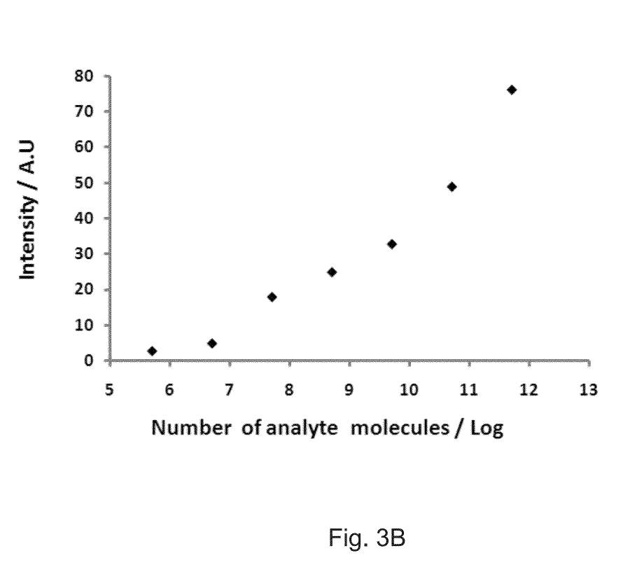

Reference is made to FIGS. 3A-3B representing a fluorescence signal as a function of analyte concentration obtained for a tube fully covered from the inside with probe molecules. The signal was measured here by a Fujifilm FLA 5100 fluorescent imaging (two-dimensional scanner). In particular, FIG. 3A shows the fluorescence signal both by the fluorescence image and by a plot of the fluorescence intensity as a function of the position along the tube. FIG. 3B shows a plot of total intensity signal versus the number of DNA molecules in the solution flown through the tube.

In this connection, it should be understood, that the concentration of the DNA is not the relevant factor for evaluating the system sensitivity but rather the number of molecules. This is because even if the concentration of DNA is very low, flowing enough solution through the tube will result in the capture of DNA molecules by the probe molecules in the tube. The length of the area at which analytes were reacted depends of course on their number and on the diameter of the tube. In this specific example, if a single DNA molecule is captured in the tube, it results in 7 chromophores that are attached to the probe by the DNA polymerase. One can realize that more chromophores can be used when needed. For example, if the detection system is not sensitive enough, the fluorescence signal can be increased per a recognition event by enlarging the signal amplification part of the DNA probe which will include more G bases, and therefore more C labels with dye will be incorporated to the sequence during the polymerization process. It must be appreciated that any method that allows the detection of the hybridization process, between the analyte and the probe, by fluorescence can be applied here.

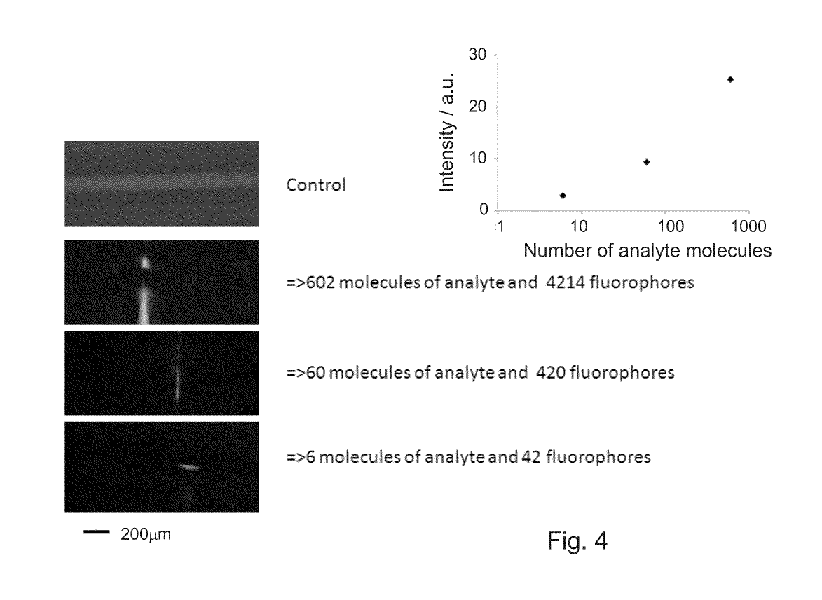

Reference is made to FIG. 4 representing the fluorescence signal of very low amount of analyte (from 602 molecules of analyte and 4214 fluorophores to from 6 molecules of analyte and 42 fluorophores). The fluorescence signal was measured by a fluorescence microscope (Olympus BX62 & BX50WI).

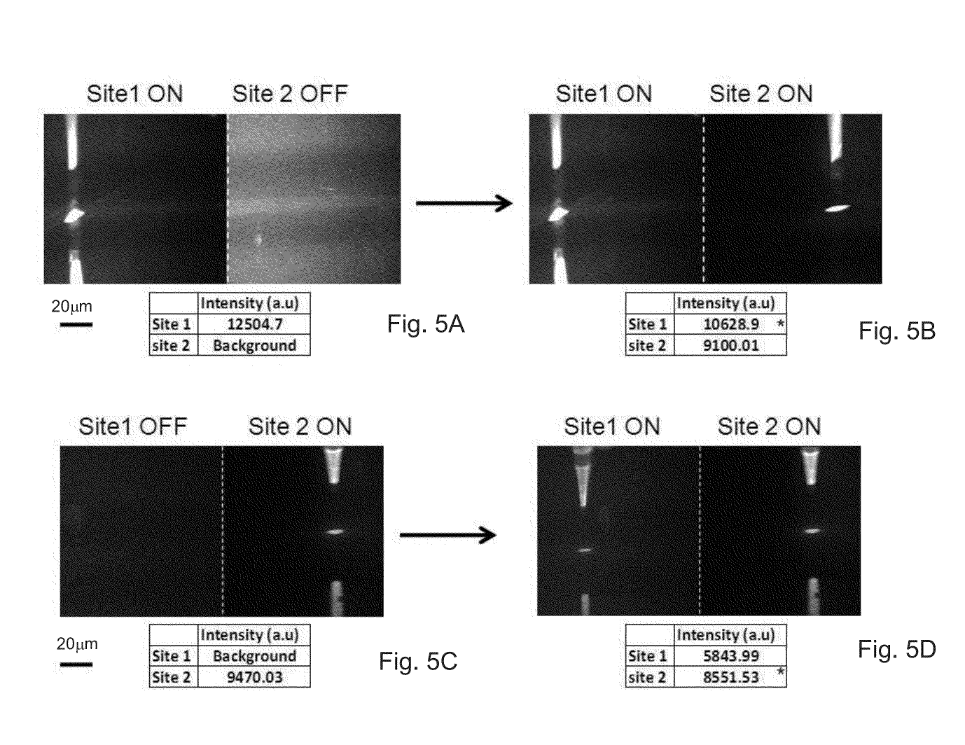

In order to appreciate the selectivity of the sensor, reference is made to FIGS. 5A-5D representing fluorescence signals obtained when the tube was modified with a DNA₁ probe and a DNA₂ probe on two sites (site 1 and site 2 respectively) in the inner surface of the tube, according to the procedure described in FIG. 1B.

The DNA sequences were selected as follows:

1. DNA₁ probe

Biotin-5′-CATGCCTGCAGGTCGACTCTAGAGGATCCCCGGGTACCG
AGCTCGAATTCG-3′

2. Analyte of DNA₁ probe

5′-CGAATTCGAGCTCGGTACCCGGGGATC-3′

3. DNA₂ probe

Biotin-5′-CATGCCTGCAGGTCGACTCTAGAGAGCTTTTAAACGTTA
GATCTTCAAGCA-3′

4. Analyte of DNA₂ probe

5′-TGCTTGAAGATCTAACGTTTAAAAGCT-3′

One of the analytes (1 or 2) was injected into the tube following by washing and application of the replication by DNA polymerase and nucleotides. After another washing the fluorescent signal was measured. The process was repeated with the second analyte. The concentration of both analytes was 10 nM and 1 μl solution was flown through the tube, namely about 6×10⁹ molecules. The distance between the probe sites was 1 mm. In particular, FIG. 5A represents the fluorescence signal from the DNA₁ probe site (site 1) after analyte 1 only was injected into the tube, following by washing and application of the replication by DNA polymerase and nucleotides.

FIG. 5B represents the fluorescence signal from the DNA₁ probe and DNA₂ probe on two sites (site 1 and site 2) after analyte 2 was also injected into the tube following by washing and application of the replication by DNA polymerase and nucleotides.

FIG. 5C represents the fluorescent signal in DNA₂ probe site (site 2) after analyte 2 only was injected into the tube following by washing and application of the replication by DNA polymerase and nucleotides

FIG. 5D represents the fluorescence signal in DNA₁ probe and DNA₂ sites (site 1 and site 2) after analyte 1 was also injected into the tube following by washing and application of the replication by DNA polymerase and nucleotides.

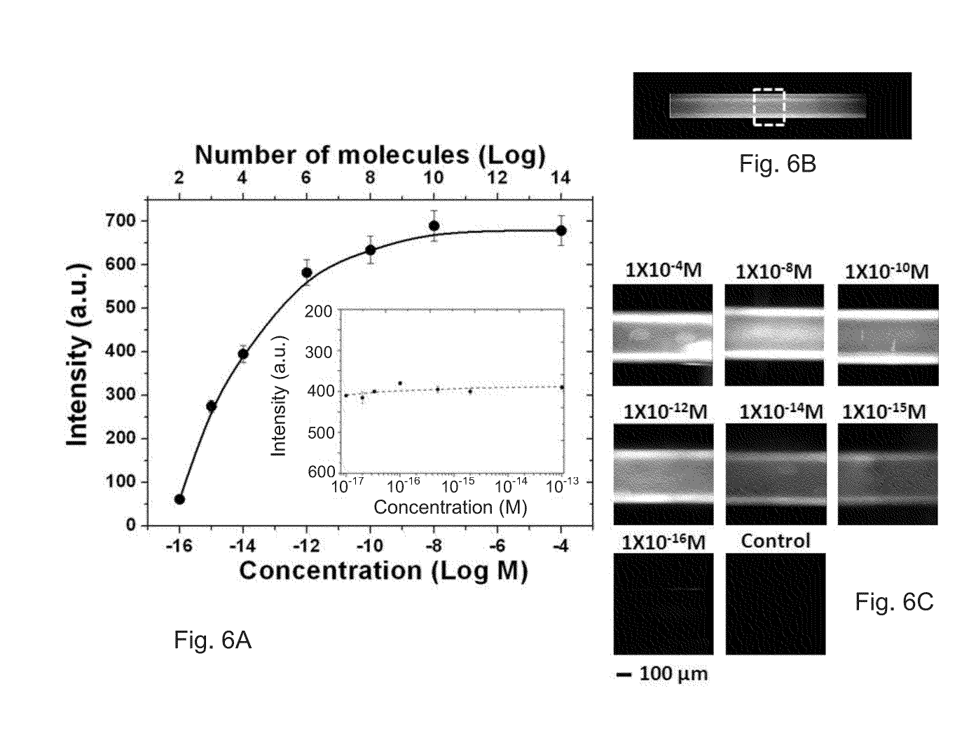

Reference is made to FIG. 6A showing the detected fluorescence signal of the DNA analyte, as a function of the DNA analyte concentration, ranging from 10⁻¹⁶ to 10⁻⁴ M (in a 1 μl sample). The fluorescence intensity is shown as a function of the number of DNA molecules spanning a concentration range of 12 orders of magnitude. The inset of FIG. 6A shows the fluorescence intensity when a constant number of molecules were detected. This was achieved by using solution with the same number of molecules but different volumes, that passed through the tube. This inset demonstrates the sensitivity of the detection to the number of molecules, not to their concentration. This was obtained by flowing different volumes of the solution through the tube keeping the same number of analyte molecules. The signal intensity did not vary even when the solution was diluted by four orders of magnitude. This indicates that the sensor detected all the analyte molecules once they were inserted to the tube, independent of the concentration of the analyte in the solution.

FIGS. 6B-6C are the actual images of the fluorescence signal for various concentrations of DNA molecules, when 1 μl of solution was passed through the tube. In this case, the entire surface of the tube was coated with the probe molecules. The error bars represent one standard deviation.

The sensitivity of the sensor of the present invention was determined by using the configuration of FIG. 1D. The number of molecules detected was determined by diluting the stock solution. The signal saturated when 10⁸ analyte molecules were injected into the tube. Below an approximate 10⁵ molecules, a linear dependence between the analyte concentration and the signal was obtained.

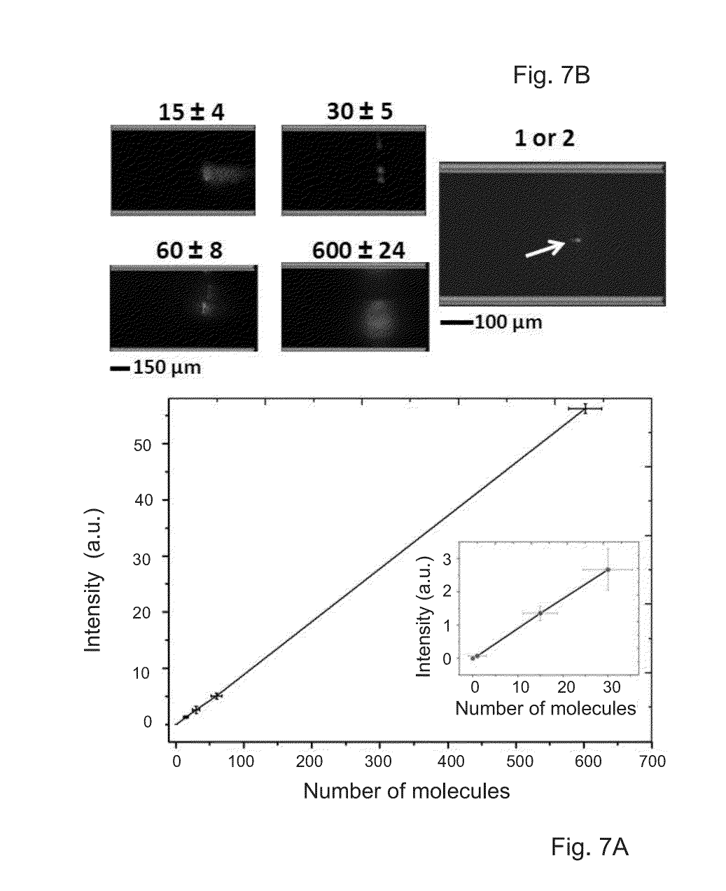

Reference is made to FIGS. 7A-7B showing a fluorescence signal of the sensor of the present invention when a small number of DNA molecules were passed through the tube. In the inset of FIG. 7A, the signal represents a range of 1 to 30 analyte molecules. The concentration of the solution containing the analytes was determined by dilution of a stock solution with a known concentration and injecting 1 μl of the diluted solution. FIGS. 7A-7B show the analysis of at least three samples with the lowest number of analyte molecules (ranging between 1 to 600 molecules). Based on the calibration curve, as well as on the statistics of the signal, the detection limit was determined to be at a single molecule level. The sensitivity of the sensor depends on the nominal number of analyte molecules and not on its concentration, because the entire solution flows through the tube, allowing all the analyte molecules to react with the surface-bound probe. Due to the small diameter of the tube, the time that the analyte molecules diffuse to the tube surface, where the probes are adsorbed, is very short (on the scale of 100 sec for 100 μm inner diameter tube). Thus, the ratio between the flow rate and the diffusion time ensures that all analyte molecules will interact with the area coated with the probe molecules.

The sensitivity for RNA detection by the sensor of the present invention was analyzed by patterning the surfaces of the inner tube with a probe as described in FIG. 1E. The fluorescence signal obtained, as a function of the RNA analyte concentration, is shown in FIG. 7A. The signal spanned a dynamic range of 10 orders of magnitude in the RNA analyte concentration until reaching saturation in the presence of 10¹⁰ analyte molecules.

The sensitivity for native mRNA detection was tested using a 20-base probe derived from the actin gene (NCU04173.4) sequence of the filamentous fungus N. crassa. The entire length of the N. crassa actin mRNA is 1448 bases and the sequence used for detection corresponded to nucleotides 296 to 316 of the mRNA. This implies that 296 bases were potentially used as a template for polymerizing the probe by RT. This sequence of 296-bases includes 48 guanine bases, which can bind to fluorescent cytosine. The tube surface was patterned with an 80-base-long probe, where the 20 bases at the 3′ end complement N. crassa actin mRNA.

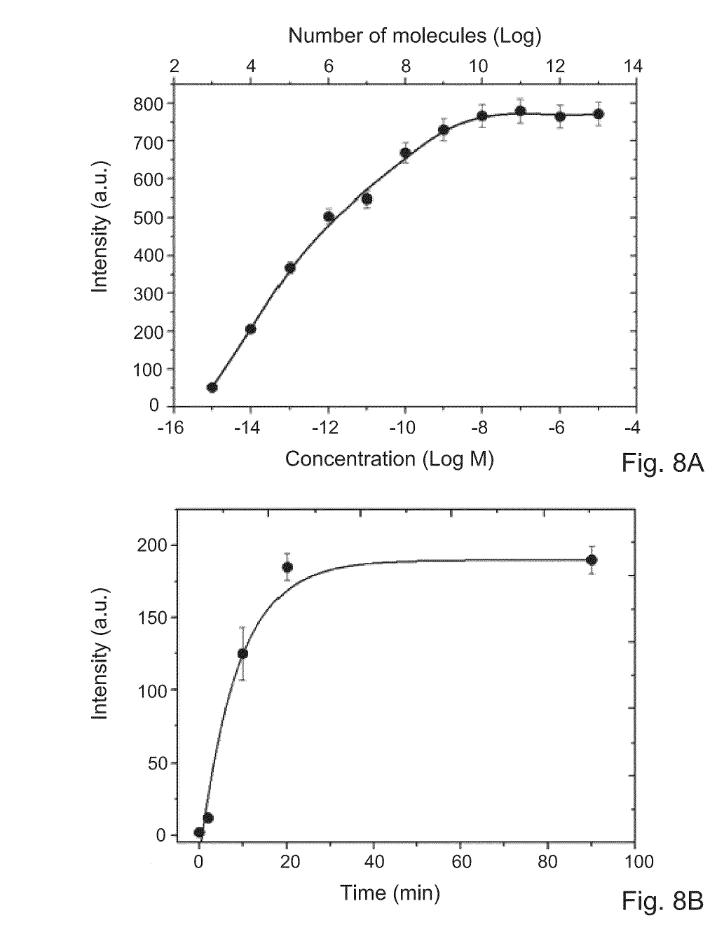

Reference is made to FIG. 8A showing a concentration-dependent curve of the RNA analyte and to FIG. 8B showing a time-dependent fluorescence signal of 10⁻¹¹ M N. crassa RNA analyte. A solution of 10⁻¹¹ M (10⁷ molecules in 1 μl) total N. crassa mRNA, which is within the linear response range of the device (FIG. 8A), was injected and the fluorescent signal saturated after about 20 minutes (FIG. 8B).

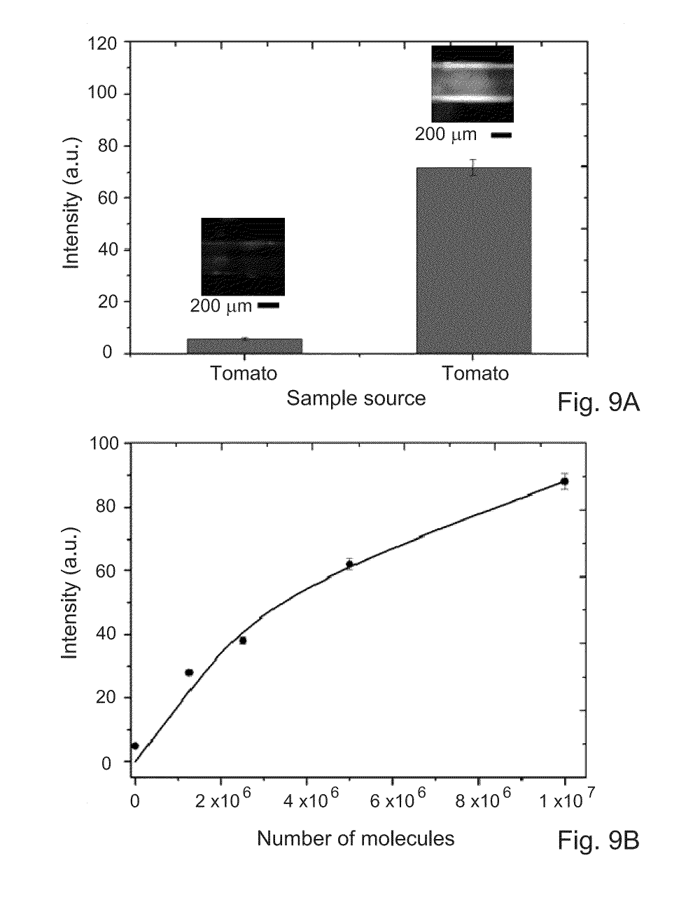

Chemical processes on surfaces may suffer from reduced selectivity due to non-specific interactions of the analyte with the surface. In the present case, interference may also occur if the nucleotides in the solution interact non-selectively with the tube's surface. To probe the specificity of the sensor, the N. crassa total mRNA preparation was mixed with a 100-fold higher concentration of total mRNA from tomato. The specificity and selectivity of the detection of mRNA is shown in FIG. 9A. FIG. 9A shows a comparison between the fluorescence signal of a sensor exposed to a 1 nM RNA solution from tomato and a mixture sample of 10 pM RNA from N. crassa with 1 nM RNA from tomato. The probe used was specific for N. crassa actin mRNA. The specific to non-specific signal ratio was about 10:1 and since the concentration of the non-specific mRNA was a hundred fold higher than that of the specific one, the specificity of the detection was better than 1000:1. FIG. 9B shows a concentration-dependent curve of sensing a N. crassa RNA analyte in a mixture containing human serum. The sensitivity and selectivity of the detection was further demonstrated when the N. crassa total mRNA was mixed with a human serum sample, which did not interfere with the detection of the specific mRNA (FIG. 9B).

Generally, for the RNA detection, a 40-base RNA oligonucleotide described above in FIG. 1E or total RNA from N. crassa described above in FIGS. 7A-7B or RNA from tomato described above in FIGS. 9A-9B was dissolved in 0.1 mM BP (pH 7.5). A reaction solution containing 20 μl of the analyte RNA, 200 units of reverse transcriptase, 0.1 M dithiothreitol, 10 μM of dNTP, and 50 μM of dCTP labeled with ATTO 495 was prepared. 1 μl of this mixture was injected to the tube and was incubated for 90 minutes at 42° C. Then, the tube was washed with BP and dried with a stream of nitrogen. The emission signal at a wavelength of 527 nm was observed and measured with a fluorescence microscope (Olympus BX62 & BX50WI).

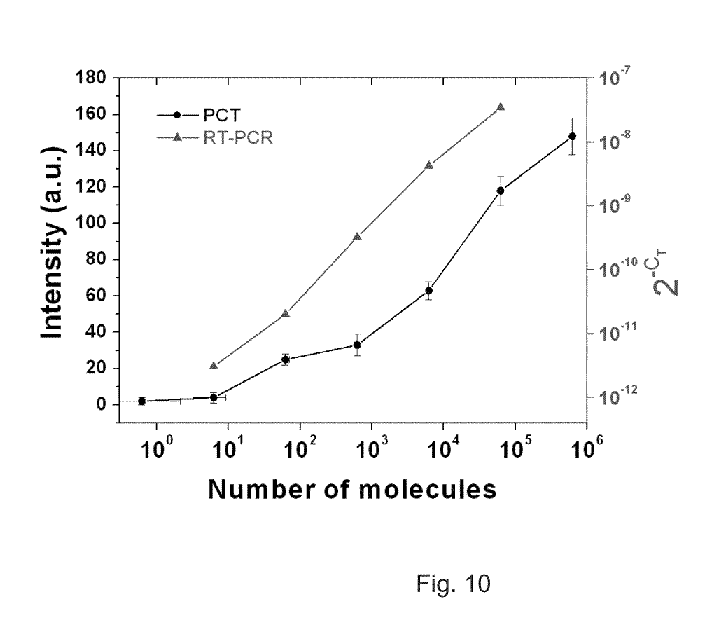

The sensitivity of the sensor of the present invention was further verified by comparison to that of real-time PCR (RT-PCR). Reference is made to FIG. 10 presenting the results obtained for the same N. crassa mRNA sample using the two methods. FIG. 10 shows a comparison between the sensor of the present invention and RT-PCR. In the obtained results of RT-PCR, CT are the threshold cycles number for detection. The mean CT was calculated from the triplicate reaction and express by 2-CT. The fluorescence signal is presented by arbitrary units. The sensitivity of the sensor was equal or better than that of RT-PCR, reaching a detection sensitivity of a single molecule. However, it is noted that on top of its sensitivity, the method of the present invention is simple to operate and does not require special sample preparation. Moreover, the time required for the analysis is independent of the analyte concentration.

In the real-time PCR array, the N. crassa total RNA was used for cDNA synthesis using the SuperScript III First-Strand Synthesis System for RT-PCR (Invitrogen) according to the manufacturer's instructions. The N. crassa actin gene (NCU04173.5) was used as target for detection [15, 16]. Absolute quantitation analysis was performed on an ABI StepOne™ Real-Time PCR Sequence Detection System and software (Applied Biosystems®), using TaqMan® Universal PCR Master Mix (Applied Biosystems®), with the primer pair actin173FP (5′-AATGGTTCGGGTATGTGCAA-3′) and actin611RP (5′-CTTCTGGCCCATACCGATCAT-3′) and TaqMan probe (5′-FAM-CAGAGCTGTTTTCCCTTCCATCGTCGGT-3′) (Applied Biosystems®), according to the manufacturer's default operating procedures.

A quantitative RT-PCR calibration curve was generated on the basis of a stepwise-diluted actin PCR amplicon, amplified from cDNA on an Eppendorf Mastercycler Gradient Thermocycler (Eppendorf), using Phusion® High-Fidelity PCR Master Mix (Finnzymes), with the primer pair actinATGF (5′-ATGGAGGAAGAAGTTGCCG-3′) and actinTAAR (5′-TTAGAAGCACTTGCGGTGC-3′) (Applied Biosystems®), isolated and purified from an agarose gel using the kit Wizard SV Gel and PCR Clean-Up System (Promega).

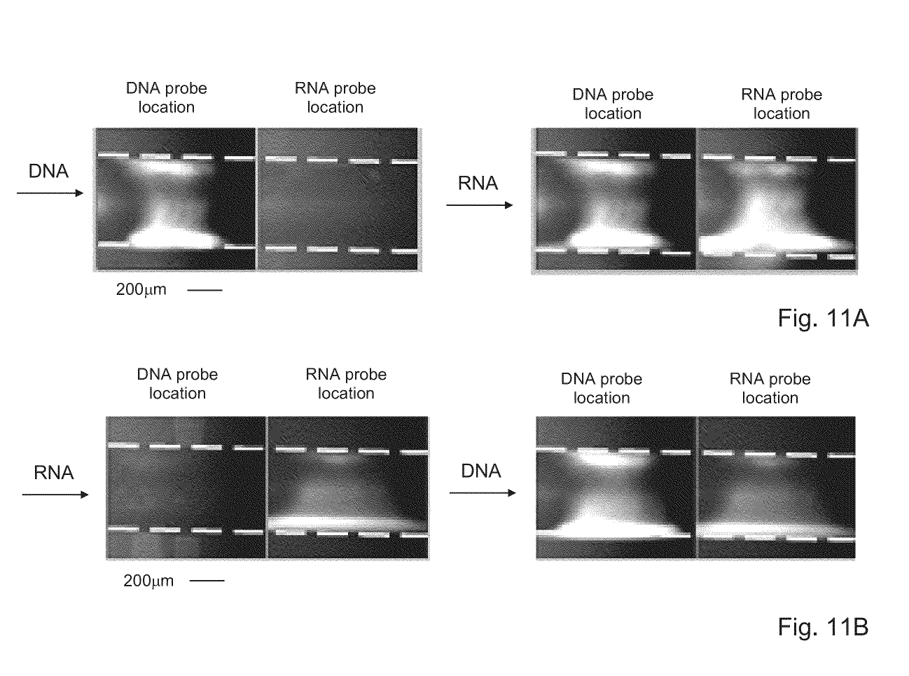

Another special feature of the sensor of the present invention is its ability to distinguish between DNA and RNA simultaneously. The selectivity of the sensor is obtained by appropriate design and application of the “developing solution”, to first include the enzyme that elongates one type of oligonucleotide and subsequently injecting another developing solution that contains the enzyme required for a different elongation process. This is demonstrated in FIGS. 11A-11B with a tube patterned with dual-probes, one for DNA and one for RNA. FIGS. 11A-11B show the specificity and selectivity of the sensor of the present invention with two different probes. A DNA analyte sample was injected into the tube, followed by a “developing” solution containing DNA polymerase with dNTPs. The DNA probe site fluoresces whereas the RNA probe site remains dark. Following an RNA analyte injection, and a developing solution containing RT with dNTPs, the RNA site fluoresces too. In a reciprocal experiment an RNA analyte was injected, followed by an RT and a dNTPs reaction mixture and a second DNA analyte was injected along with the DNA polymerase/dNTPs reaction mixture. In this experiment, the site containing the RNA probe fluoresced first and only following the injection of a DNA developing, both locations show a fluorescence signal. In this specific and non-limiting example, an analyte solution containing 1 pM of DNA was first injected through the tube, followed by a solution containing DNA polymerase and dNTPs. At this stage, the DNA probe was elongated, while the RNA probe did not incorporate fluorescent chromophores. Following an injection of a solution of 1 pM RNA and the addition of RT with dNTPs, the RNA probe was elongated and fluorescence was observed at the two sites. The reciprocal experiment was performed with another patterned tube. The RNA analyte was injected, followed by RT and dNTPs, resulting in RNA elongation and a fluorescence signal appearing at the RNA-probe site on the tube. During the second stage, the DNA analyte, followed by DNA polymerase and dNTPs, were injected and fluorescence appeared at the site of the DNA probe.

The sensor of the present invention enables quantitative detection of a few analyte molecules, both DNA and RNA simultaneously. The novel method can detect both DNA and RNA in the same measurement, so that quantification of both in the same sample is reliable and improves the quality of the entire detection process. This may allow detection of gene expression in the same analyzed medium.

It should be noted that by using the sensor of the present invention, no pretreatment required. This feature may be of special importance in the case of small oligomers like microRNA. Moreover, the sensor of the present invention allows simultaneous detection of several DNA/RNA molecules. Furthermore, it is possible to release analytes after they have been hybridized and detected, to be used for further investigation.

The results indicate that in addition to extremely high sensitivity, the sensor of the present invention is also robust, even in the presence of potentially interfering elements, as indicated in the measurements performed on mRNA in the presence of human serum (FIG. 9B) without a need for to pretreat the analyte solution.

Moreover, it should be noted that in methods based on probes immobilized on a two-dimensional surface, the detection time is inversely proportional to the concentration of the analyte because the analyte molecules must diffuse on the surface until they interact with the probe “pixel”. In the sensor of the present invention, the analyte flows through the tube and the relevant diffusion time is related to the diffusion in the transverse direction. Hence, tubes with smaller diameters enable faster detection (for example ranging from 5 to 30 min). However, the smaller diameter limits the amount of solution that can flow through the tube at a given time. The short detection time and the sensitivity of the system to the number of analytes and not to their concentration makes this system efficient and ultra sensitive. These properties are the result of flowing the entire sample through the tube. Additional analysis, however, is required to optimize the conditions of the sensor of the present invention so that the detection time is further reduced and the selectivity is improved.

Moreover, the sensor of the present invention has a simple calibration. By calibrating the signal of the fluorescence intensity, it is possible to detect and quantitatively analyze oligonucleotides within a very large dynamic range, from nanomoles to a single molecule. The extent of its selectivity depends, like in many other methods, on the conditions of the hybridization, namely, temperature and pH.

Furthermore, the sensor of the present invention does not require expensive equipment. The patterned tube can be produced automatically and therefore tubes patterned with up to few tens of different probes are simple and inexpensive to produce. The detection system is also simple and compact, mechanically stable, and mobile. The sensor of the present invention can be combined with microfluidic set-up that allows, for example, circulating the analyte solution several times through the tube to ensure detection of even minute amount of molecules. The number of probes can be expanded by connecting tubes in an arrayed structure so that the same analyte solution flows through many tubes. This configuration is similar to a two-dimensional array, but here the analyte solution flows from one “pixel” to another, significantly reducing the required detection time. Due to its simplicity, the sensor of the present invention is expected to cost a fraction of the cost of real-time PCR machines and it can be operated by “non-experts”.

The sensor of the present invention based on patterning the inner surface of a capillary tube in a simple and inexpensive way, opens up the possibility for quantitative detection and identification of oligonucleotides in a new sensitive, mobile, simple-to-operate, inexpensive DNA/RNA detection system. The mobility of the detection enables new applications of DNA and RNA analysis in medicine and environmental applications.

1-10. (canceled)

11. A method for use in detection of one or more analytes contained in a fluid sample, the method comprising:
providing an array of surface regions of a flow cavity carrying different probes at fixed locations along said cavity, said providing comprising patterning an inner surface of a tube-like member, a pattern being in the form of an array of spaced-apart locations comprising locations carrying different probes immobilized to the inner surface of the tube-like member; said different probes comprising different sequence-specific biological substances such that each probe is configured and operable to react with a specific analyte in the sample while flowing through said cavity;
said patterning comprises immobilizing a plurality of different substances corresponding to different probes respectively to spaced-apart locations along the inner surface of the tube-like member, said immobilizing comprising: (a) patterning the inner surface of the tube-like member to create an array of locations functionalized for binding to the different substances and spaced by regions of the inner surface incapable of any such binding; (b) flowing a substance of a first type, functionalized for binding to said locations, while applying a magnetic field pattern to said cavity such as to permit binding of said substance of said first type to one or more specific locations and prevent the binding to other of said locations; (c) repeating step (b) a predetermined number of times, each time modulating the magnetic field pattern for binding another type of said plurality of substances to another one or more locations, until binding all the substances from said plurality of substances to different locations; flowing said fluid sample along said flow cavity and causing successive interactions between said array of the surface regions of said cavity, thereby resulting in different surface modifications of the cavity; and;
detecting data indicative of the surface modification and selectively and specifically identifying at least one analyte molecule quantitatively in accordance with the one or more fixed locations of interaction corresponding to the surface modification.

12. The method of claim 11, wherein said detecting and identifying depends on the number of molecules of said analyte in the sample and is independent of a concentration of the analyte in the sample.

13. The method of claim 11, wherein said detecting and identifying comprises analyzing a radiation response from one or more of the probes and determining one or more of the probe locations where said response have been originated as a result of said reacting.

14. The method of claim 1, wherein said reacting comprises hybridization between a biological moiety of the probe and the corresponding analyte.

15. The method of claim 14, wherein said detecting and identifying of the one or more analytes comprises flowing through said cavity at least one replication biological moiety marked with one or more fluorescent labels respectively.

16. The method of claim 11, wherein the different probes comprises different substances.

17. The method of claim 11, comprising providing desired sensitivity of the detection and identification of the analyte in the sample by controlling at least one of the following parameters: a geometrical parameter of said flow cavity, a dimension of a feature of said probes, and a parameter of said fluid sample flowing through the flow cavity.

18. (canceled)

19. The method of claim 11, wherein said different biological substances comprises biological moieties.

20. The method of claim 19, wherein said biological moieties comprise at least one of DNA sequence, RNA sequence, antibodies, antigens and proteins.

21. The method of claim 20, comprising detecting and identifying at least one DNA sequence and at least one RNA sequence simultaneously.

22. The method of claim 11, wherein said selectively identification of said analyte further comprises flowing a first developing solution along said flow cavity including a biological substance elongating one type of analyte and subsequently injecting another developing solution along said flow cavity containing another biological substance required for a different elongation process.

23. (canceled)

24. The method of claim 11, wherein said patterning of the inner surface of the tube-like member comprises:
applying a first modulated magnetic field in the vicinity of said cavity thus creating a certain pattern of the plurality of locations of interaction to be obtained on the inner surface of said tube-like member;
interacting said inner surface of said tube-like member with magnetic particles under the application of said first modulated magnetic field, thereby attracting the magnetic particles to said locations of interaction while being substantially not attracted to spaces between said locations of interaction, thus creating in the inner surface of said tube-like member said certain pattern of locations interacted with the magnetic particles;
flowing a first reacting agent through the tube-like member thereby interacting said first reacting agent via chemical recognition and/or biological recognition with said inner surface of said tube-like member within spaces between said locations and binding said first reacting agent with the inner surface in said spaces, while said magnetic particles blocking the binding of said first reacting agent to said locations;
removing at least a portion of the magnetic particles by removing an effect of the magnetic field, resulting in a negatively patterned inner surface;
flowing a second reacting agent through the tube-like member thereby binding said second reacting agent via chemical recognition and/or biological recognition with said inner surface of said tube-like member at said locations free of the first reacting agent bonded to said inner surface of said tube-like member, to create said array of locations functionalized for binding to the probe substances;
applying a second modulated magnetic field in the vicinity of said cavity configured to define a predetermined number of said locations to be excluded from interaction with one or more of the probe substances; and;
interacting said inner surface of said tube-like member with magnetic particles under the application of said second magnetic field, thereby attracting the magnetic particles to said predetermined number of locations, thus blocking said predetermined number of locations from interaction with said substances.

25. The method of claim 11, wherein the modulated magnetic field is applied by a magnetic pattern generator defining an arrangement of ferromagnetic material at identifiable well-defined positions corresponding to well defined locations along said tube-like member.

26. The method of claim 11, wherein said interacting of said inner surface of said tube-like member with magnetic particles comprises flowing a solution containing the magnetic particles through the cavity such that the particles are attracted towards the identifiable well-defined positions of the magnetic pattern generator.
